(19) United States
(12) Patent Application Publication
(10) Pub. No.: US 2008/0142759 A1
(43) Pub. Date: Jun. 19, 2008

(54) Pyridine N-Oxide Based Azo Dyes And Their Metal Complexes For Use In Optical Layers For Optical Data Recording

(76) Inventor: Christophe Pays, Rixheim (FR)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/792,867

(22) PCT Filed: Dec. 7, 2005
(86) PCT No.: PCT/EP05/56559

§ 371 Date: Jun. 8, 2007

(30) Foreign Application Priority Data
Dec. 8, 2004 (EP) 04029031.4

(51) Int. Cl. C09K 11/06; C07D 239/24; C07F 15/00; C07F 15/04

(52) U.S. Cl. 252/301.16; 544/300; 544/225; 427/162

(57) ABSTRACT

The present invention relates to the use of pyridine N-oxide based azo dyes and their metal complexes in optical layers for optical data recording, preferably for optical data recording using a blue laser with a wavelength up to 450 nm or a red laser with a wavelength up to 650 nm.

The invention further relates to a write only read many (WORM) type optical recording medium capable of recording and reproducing information with radiation of a blue or red laser, which employs a pyridine N-oxide based azo dye or a respective metal complex in the optical layer.

The present invention relates to the use of pyridine N-oxide based azo dyes and their metal complexes in optical layers for optical data recording, preferably for optical data recording using a blue laser with a wavelength up to 450 nm or a red laser with a wavelength up to 650 nm.

The invention further relates to a write only read many (WORM) type optical recording medium capable of recording and reproducing information with radiation of a blue or red laser, which employs a pyridine N-oxide based azo dye or a respective metal complex in the optical layer.

Recently, organic dyes have attracted considerable attentions in the field of diode-laser optical storage. Commercial recordable compact discs (CD-R) and recordable digital versatile discs (DVD-R) can contain, as recording layer, numerous dyes based on phthalocyanine, hemicyanine, cyanine and metallized azo structures. These dyes are suitable in their respective fields with the laser wavelength criteria. Other general requirements for dye media are strong absorption, high reflectance, high recording sensitivity, low thermal conductivity as well as light and thermal stabilities, durability for storage or non-toxicity.

For industrial application, these dyes have to be suitable for the spin coating process to prepare thin films, i.e. they have to be sufficiently soluble in the organic solvents generally applied in the spin coating process.

WORM (write once read many) type and erasable type optical recording media reproduce information by detecting variations in the reflectivity caused by physical deformation, by alterations of optical characteristics as well as by phase and magnetic properties of a recording layer before and after the recording.

Recordable compact discs (CD-R) are widely known as a WORM type optical recording medium. Recently, digital versatile discs (DVD-R) with increased information storage capabilities up to 4.7 GBytes have been commercialized.

The DVD-R technology adopts as a light source a red diode laser with a wavelength of 630-670 nm. Thereby the pit size and track interval can be reduced, increasing the information storage capacity by up to 6-8 times compared to CD-R's.

Blu-ray® discs (Blu-ray® disc is a standard developed by Hitachi Ltd., LG Electronics Inc., Matsushita Electric Industrial Co. Ltd., Pioneer Corporation, Royal Philips Electronics, Samsung Electronics Co. Ltd., Sharp Corporation, Sony Corporation, Thomson Multimedia) are going to be the next milestone in optical recording technology. Its new specification increases the data storage up to 27 GBytes per recording layer for a 12 cm diameter disc. By adopting a blue diode laser with a wavelength of 405 nm (GaN or SHG laser diodes), the pit size and track interval can be further reduced, again increasing the storage capacity by an order of magnitude.

The construction of optical data recording media is known in the art. An optical data recording media generally comprises a substrate and a recording layer, the optical layer. Usually discs or wavers of organic polymeric materials are used as substrates. Preferred substrates are polycarbonate (PC) or polymethylmethacrylate (PMMA). The substrate has to provide an even and uniform surface of high optical quality. The optical layer is deposited thereon in a thin and uniform film of high optical quality and defined thickness. Finally, a reflective layer, e.g. aluminium, gold or copper, is deposited upon the optical layer.

Advanced optical data recording media may comprise further layers, such as protective layers, adhesive layers or additional optical layers.

To provide for a thin and uniform film of the optical layer, the material is usually deposited by spin coating, vacuum evaporation, jet coating, rolling coating or soaking. The preferred process in industry is spin coating to form an optical layer of about 70 nm to 250 nm thickness. For the application in the spin coating process, the material of the optical layer has to be highly soluble in organic solvents.

Pyridine N-oxide based azo dyes are known for many years. For example, a dye of the structure below has been disclosed to have very good affinity for cellulose acetate fibers (U.S. Pat. No. 3,249,597).

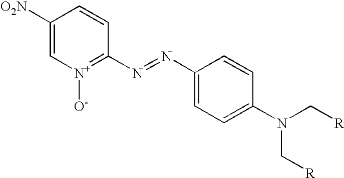

Such pyridine N-oxide based azo dyes have also been disclosed for use in hair dyeing compositions (see for example U.S. Pat. No. 3,955,918).

Metal complexes, in particular copper(II), nickel(II), cobalt(II), iron(III) and manganese(II) complexes of pyridine N-oxide based azo compounds are described in the following publications: a) Renko, D.; Koprivanac, N.; Jovanovic-Kolar, J.; Osterman, D. Kemija u Industriji (1979), 28 (2), 53-58. b) Koprivanac, N; Jovanovic-Kolar, J.; Kramer, V. International Journal of Mass Spectrometry and Ion Physics (1983) 47, 531-534. Formation of such metal complexes are used in the spectrophotometric determination of iron, copper, scandium and zirconium. For iron and copper, see: Beaupre, P. W.; Holland, W. J. Mikrochimica Acta (1983) 3(1-2) 71-75. For scandium see: Beaupre, P. W.; Holland, W. J. Mikrochimica Acta (1982) 2(5-6) 419-422. For zirconium see: Beaupre, P. W.; Holland, W. J. Mikrochimica Acta (1980) 2(1-2) 53-57.

Surprisingly it has now been found, that specific pyridine N-oxide based azo dyes and their metal complexes as described below are useful as dye compounds in optical layers for optical data recording media.

The present invention therefore relates to the use of pyridine N-oxide based azo dyes and their metal complexes in an optical layer as described below and to the use of said optical layers for optical data recording media.

More particularly, the invention relates to a write only read many (WORM) type optical data recording medium capable of recording and reproducing information with radiation of blue laser of preferably 405-410 nm or with radiation of a red laser of preferably 635-640 nm, which employs a pyridine N-oxide based azo dye and or a respective metal complex in the optical layer.

The present invention is directed to the use of a dye compound of formula (I) or (II) in an optical layer for optical data recording

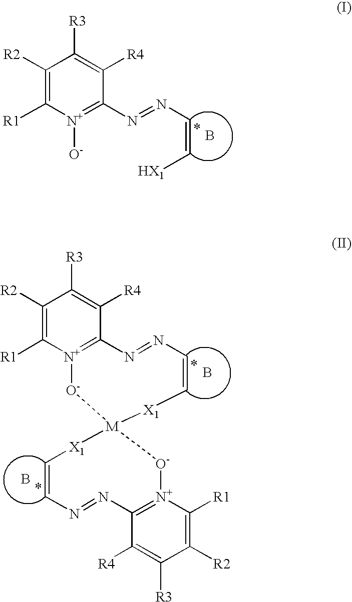

wherein
R₁ to R₄ independently of one another, represent hydrogen, cyano (—CN), halogen (F, Cl, Br, I), nitro (NO₂), hydroxy (—OH);
   C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkenyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
   C₁₋₈ alkoxy (—OR) wherein the alkyl (R) can be unsubstituted or substituted by C₁₋₈ alkyl, C₁₋₈ alkenyl, hydroxy (—OH), by C₆₋₁₂ aryl or by —NR₅R₆ in which R₅ and R₆ are independently hydrogen, C₁₋₈ alkyl, C₁₋₈ alkenyl or C₆₋₁₂ aryl;
   —CX₃ where X can be chlorine, fluorine, bromine;
   —NR₅R₆ in which R₅ and R₆ are independently hydrogen, C₁₋₈ alkyl, C₁₋₈ alkenyl or C₆₋₁₂ aryl;
   C₁₋₈ alkylthio (—SR), wherein the alkyl (R) can be unsubstituted or substituted by C₁₋₈ alkyl, hydroxy (—OH), by C₆₋₁₂ aryl or by —NR₅R₆ in which R₅ and R₆ are independently hydrogen, C₁₋₈ alkyl or C₆₋₁₂ aryl;
   C₁₋₈ sulfoxide (—S(O)R), wherein the alkyl (R) can be unsubstituted or substituted by C₁₋₈ alkyl, hydroxy (—OH), by C₆₋₁₂ aryl or by —NR₅R₆ in which R₅ and R₆ are independently hydrogen, C₁₋₈ alkyl or C₆₋₁₂ aryl;
   C₁₋₈ sulfone (—SO₂R), wherein the alkyl (R) can be unsubstituted or substituted by C₁₋₈ alkyl, hydroxy (—OH), by C₆₋₁₂ aryl or by —NR₅R₆ in which R₅ and R₆ are independently hydrogen, C₁₋₈ alkyl or C₆₋₁₂ aryl;
   C₁₋₈ sulfonamide (—SO₂NR₅R₆), in which R₅ and R₆ are independently hydrogen, C₁₋₈ alkyl or C₆₋₁₂ aryl;
   C₁₋₈ carbonamide (—CO₂NR₅R₆), in which R₅ and R₆ are independently hydrogen, C₁₋₈ alkyl or C₆₋₁₂ aryl;
   C₁₋₈ phosphoramide (—P(O)(NR₅R₆)₂), in which R₅ and R₆ are independently hydrogen, C₁₋₈ alkyl or C₆₋₁₂ aryl;
   C₁₋₈ phosphate (—P(O)(OR)₂), in which R represent C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
X₁ represents oxygen, sulphur, selenium,
   —NR in which R represent hydrogen, cyano, C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
   —NSO₂R in which R represent C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
   —NP(O)(OR)₂ in which R represent C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
B represent an unsubstituted or a substituted aromatic ring; or
   a five or a six-membered heterocyclic ring of the following structures:

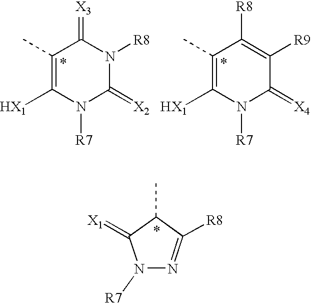

wherein
R₇ to R₉ independently of one another, represent hydrogen, halogen, cyano, C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
   —SO₂R wherein R represent C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
X₂ to X₃ represent oxygen, sulphur, selenium,
   —NR in which R represent hydrogen, cyano, C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
   —NSO₂R in which R represent C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
   —NP(O)(OR)₂ in which R represent C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
X₄ represent oxygen, sulphur, selenium;
   ═NR in which R represent hydrogen, cyano, C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl; or ═C(CN)₂;
M represent a metal atom including Al, In, Sn, Ti, V, Cr, Mn, Fe, Co, Ni, Cu, Zn, Zr, Ru, Rh, Pd, Cd, Hf, Re, Os, Ir, Pt, Hg,

In a preferred aspect, the present invention is directed to a dye compound of formula (II), wherein the dye compound of formula (II) is of the more specific formula (III)

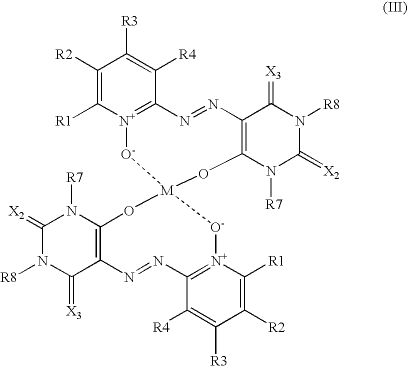

M is selected from the group consisting of Ni, Cu, Co, Zn, Al, Fe, Ru, Pd, Pt, Cr, Mn;
R₁ is selected from H, Cl, CH₃, C₂H₅, C₃H₇ or unsubstituted phenyl,
R₂ is selected from H, Br, Cl, F, NR₂ wherein the alkyl (R) can be unsubstituted or substituted by C₁₋₈ alkyl, hydroxy (—OH), by C₆₋₁₂ aryl or by —NR₇R₈ in which R₇ and R₈ are independently hydrogen, C₁₋₈ alkyl or C₆₋₁₂ aryl; NO₂, CH₃, C₂H₅,
R₃ is selected from H, Cl, CH₃, C₂H₅,
R₄ is hydrogen, CH₃, C₂H₅, OR wherein the alkyl (R) can be unsubstituted or substituted by C₁₋₈ alkyl, hydroxy (—OH), by C₆₋₁₂ aryl or by —NR₇R₈ in which R₇ and R₈ are independently hydrogen, C₁₋₈ alkyl or C₆₋₁₂ aryl;
R₇ to R₈ independently of one another, represent hydrogen, C₁₋₈ alkyl, alkenyl or alkynyl, C₃₋₁₀ cycloalkyl or cycloalkenyl wherein the alkyl groups can be unsubstituted or substituted by C₁₋₈ alkyl, C₁₋₈ alkoxy, halogen, hydroxy (—OH), by C₆₋₁₂ aryl or by —NR₅R₆ in which R₅ and R₆ are independently hydrogen, C₁₋₈ alkyl or C₆₋₁₂ aryl;
X₂ to X₃ is selected from O, S, NR wherein R represent hydrogen, cyano, C₁₋₈ alkyl.

In a more preferred aspect, the present invention is directed to a dye compound of formula (III), wherein
M is selected from the group consisting of Ni, Cu, Co, Zn, Al, Fe, Cr, Mn;
R₁ is selected from H, CH₃,
R₂ is selected from H, Br, Cl, NO₂, CH₃,
R₃ is hydrogen,
R₄ is selected from H, Cl, CH₃, OC₂H₅, OH,
R₇ to R₈ independently of one another, are selected from CH₃, C₂H₅, C₃H₇ (n- or i-propyl), C₄H₉ (n-, sek- or tert.-butyl),
X₂ is selected from O, S,
X₃ is O.

In a most preferred embodiment, the present invention is directed to a dye compound of formula (III) wherein M is selected from nickel, copper or zinc, R₁ is H, R₂ is Br, R₃ is hydrogen, R₄ is OC₂H₅, R₇ and R₈ are C₂H₅, and X₂ is S, X₃ is O.

The present invention further relates to an optical layer comprising a dye compound of formula (I) or (II) as described above and to the use of said optical layer for optical data recording media. An optical layer according to the invention may also comprise a mixture of two or more, preferably of two dye compounds of formula (I) and/or (II) as defined above.

The pyridine N-oxide based azo dye compounds of formula (I) or (II) provide for particularly preferable properties when used in optical layers for optical data recording media according to the invention.

Further, the invention relates to a method for producing optical layers comprising the following steps
(a) providing a substrate
(b) dissolving a dye compound or a mixture of dye compounds of formula (I) or (II) in an organic solvent to form a solution,
(c) coating the solution (b) on the substrate (a);
(d) evaporating the solvent to form a dye layer.

Preferred substrates are polycarbonate (PC) or polymethylmethacrylate (PMMA). Organic solvents are selected from C₁₋₈ alcohol, halogen substituted C₁₋₈ alcohols, C₁₋₈ ketone, C₁₋₈ ether, halogen substituted C₁₋₄ alkane, or amides.

Preferred C₁₋₈ alcohols or halogen substituted C₁₋₈ alcohols are for example methanol, ethanol, isopropanol, diacetone alcohol (DAA), 2,2,3,3-tetrafluoropropanol, trichloroethanol, 2-chloroethanol, octafluoropentanol or hexafluorobutanol.

Preferred C₁₋₈ ketones are for example acetone, methylisobutylketone, methylethylketone, or 3-hydroxy-3-methyl-2-butanone.

Preferred halogen substituted C₁₋₄ alkanes are for example chloroform, dichloromethane or 1-chlorobutane.

Preferred amides are for example dimethylformamide or dimethylacetamide.

The optical layer (dye layer) obtained preferably has a thickness from 70 to 250 nm.

In a preferred aspect, the present invention provides for an optical layer suitable for high-density recording material, e.g. of the WORM disc format, in a laser wavelength range of from 350-450 nm, preferably around 405 nm. The present invention provides also for an optical layer suitable for high-density recording material, e.g. of the WORM disc format, in a laser wavelength range of from 630-650 nm, preferably around 635 nm. The optical layer for blue laser application being preferred.

The dye compounds of formula (I) and (II) possess the required optical characteristics (such as high absorbtivity, high recording sensitivity as example), an excellent solubility in organic solvents, an excellent light stability and a decomposition temperature of 250-350° C.

### Preparation of the Monoazo Dye (I)

The coupling reaction may be carried out in aqueous and non-aqueous solvents. Non-aqueous solvents are alcohols such as methanol, ethanol, propanol, butanol, pentanol, etc., dipolar aprotic solvents such as DMF, DMSO, NMP and water-immiscible solvents such as toluene or chloro-benzene.

The coupling is preferably carried out in a stoichiometric ratio of coupling component and diazo component. The coupling is generally done at temperatures between −30° C. to 100° C., preference being given to temperatures of −10° C. to 30° C., and particular preference to temperatures of −5° C. to 10° C.

The coupling may be carried out in an acidic as well as an alkaline medium. Preference is given to pH <10, particular preference to pH <7.0, very particular preference to pH <5.0.

### Preparation of the Metal Complexes (II)

Preferably, the complexes are prepared by reaction of a solution of one equivalent of a metal salt with a boiling solution of two equivalents of the corresponding dye in a polar solvent selected from the list below. The precipitate is isolated following standard methods.

The solvents used in the process are preferably selected from the group consisting of C₁₋₈ alcohols, alkylnitriles, aromatics, dimethylformamide, N-methylpyrrolidone or a mixture of one of these solvents with water or water itself.

Most preferred solvents used in the process are C₁₋₈ alcohols.

### Preparation of an Optical Layer

An optical layer according to the invention comprises a pyridine N-oxide azo based dye of formula (I) or (II) or a mixture of metal complexes of formula (I) and/or (II).

A method for producing an optical layer according to the invention comprises the following steps
(a) Providing a substrate
(b) Dissolving a dye compound (I) or (II) or a mixture of dye compounds of formula (I) and/or (II) in an organic solvent to form a solution,
(c) Coating the solution (b) on the substrate (a);
(d) Evaporating the solvent to form a dye layer.

Preparing of the high density optical recording medium

A method for producing an optical recording medium comprising an optical layer according to the invention comprises the following additional steps
(e) sputtering a metal layer onto the dye layer
(f) applying a second polymer based layer to complete the disk.

A high-density data storage medium according to the invention therefore preferably is a recordable optical disc comprising: a first substrate, which is a transparent substrate with grooves, a recording layer (optical layer), which is formed on the first substrate surface using the dyes compounds of formula (I) or (II), a reflective layer formed on the recording layer, a second substrate, which is a transparent substrate with grooves connected to the reflective layer with an attachment layer.

The dyes compounds of formula (I) or (II) in the form of a solid film have a high refractive index at the longer wavelength flank of the absorption band, which preferably achieves a peak value of from 2.0 to 3.0 in the range of from 350 to 500 nm for blue laser use or in the range of 500-630 nm for red laser use. The dyes compounds of formula (I) or (II) allow providing a medium having high reflectivity as well as high sensitivity and good playback characteristics in the desired spectral range.

### (a) Substrate

The substrate, which functions as support for the layers applied thereto, is advantageously semi-transparent (T>10%) or preferably transparent (T>90%). The support can have a thickness of from 0.01 to 10 mm, preferably from 0.1 to 5 mm. Suitable substrates are, for example, glass, minerals, ceramics and thermosetting or thermoplastic plastics. Preferred supports are glass and homo- or co-polymeric plastics. Suitable plastics are, for example, thermoplastic polycarbonates, polyamides, polyesters, polyacrylates and polymethacrylates, polyurethanes, polyolefins, polyvinyl chloride, polyvinylidene fluoride, polyimides, thermosetting polyesters and epoxy resins.

The most preferred substrates are polycarbonate (PC) or polymethylmethacrylate (PMMA).

The substrate can be in pure form or may also comprise customary additives, for example UV absorbers or dyes, as proposed e.g. in JP 04/167239 as light-stabilizers for the recording layer. In the latter case it may be advantageous for the dye added to the support substrate to have an absorption maximum hypso-chromically shifted relative to the dye of the recording layer by at least 10 nm, preferably by at least 20 nm.

The substrate is advantageously transparent over at least a portion of the range from 350 to 700 nm, so that it is permeable to at least 90% of the incident light of the writing or readout wavelength.

### (b) Organic Solvents

Organic solvents are selected from C₁₋₈ alcohol, halogen substituted C₁₋₈ alcohols, C₁₋₈ ketone, C₁₋₈ ether, halogen substituted C₁₋₄ alkane, or amides.

Preferred C₁₋₈ alcohols or halogen substituted C₁₋₈ alcohols are for example methanol, ethanol, isopropanol, diacetone alcohol (DAA), 2,2,3,3-tetrafluoropropanol, trichloroethanol, 2-chloroethanol, octafluoropentanol or hexafluorobutanol.

Preferred C₁₋₈ ketones are for example acetone, methylisobutylketone, methylethylketone, or 3-hydroxy-3-methyl-2-butanone.

Preferred halogen substituted C₁₋₄ alkanes are for example chloroform, dichloromethane or 1-chlorobutane.

Preferred amides are for example dimethylformamide or dimethylacetamide.

### (c) Recording Layer

The recording layer (optical layer) is preferably arranged between the transparent substrate and the reflecting layer. The thickness of the recording layer is from 10 to 1000 nm, preferably from 30 to 300 nm, especially about 80 nm, for example from 60 to 120 nm.

The use of dyes compounds of formula (I) or (II) results in advantageously homogeneous, amorphous and low-scattering recording layers having a high refractive index. The absorption edge is surprisingly steep even in the solid phase. Further advantages are high light stability in daylight and under laser radiation of low power density with, at the same time, high sensitivity under laser radiation of high power density, uniform script width, high contrast, and also good thermal stability and storage stability.

The recording layer, instead of comprising a single compound of formula (I) or (II), may also comprise a mixture of such compounds according to the invention. By the use of mixtures, for example mixtures of isomers or homologues as well as mixtures of different structures, the solubility can often be increased and/or the amorphous content improved.

For a further increase in stability it is also possible, if desired, to add known stabilizers in customary amounts, for example a nickel dithiolate as light stabilizer, as described in JP 04/025493.

The recording layer comprises a compound of formula (I) or (II) or a mixture of such compounds preferably in an amount sufficient to have a substantial influence on the refractive index, for example at least 30% by weight, more preferably at least 60% by weight, most preferably at least 80% by weight.

Further customary components are, for example other chromophores (for example those disclosed in WO-01/75873, or others having an absorption maximum at from 300 to 1000 nm), stabilizers, ¹0₂-, triplet- or luminescence quenchers, melting-point reducers, decomposition accelerators or any other additives that have already been described in optical recording media. Preferably, stabilizers or fluorescence-quenchers are added if desired.

When the recording layer comprises further chromophores, they may in principle be any dye that can be decomposed or modified by the laser radiation during the recording, or they may be inert towards the laser radiation. When the further chromophores are decomposed or modified by the laser radiation, this can take place directly by absorption of the laser radiation or can be induced indirectly by the decomposition of the compounds of formula (I) or (II) according to the invention, for example thermally.

Naturally, further chromophores or colored stabilizers may influence the optical properties of the recording layer. It is therefore preferable to use further chromophores or coloured stabilizers, the optical properties of which conform as far as possible to those of the compounds formula (I) or (II) or are as different as possible, or the amount of further chromophores is kept small.

When further chromophores having optical properties that conform as far as possible to those of compounds formula (I) or (II) are used, preferably this should be the case in the range of the longest-wavelength absorption flank. Preferably the wavelengths of the inversion points of the further chromophores and of the compounds of formula (I) or (II) are a maximum of 20 nm, especially a maximum of 10 nm, apart. In that case the further chromophores and the compounds of formula (I) or (II) should exhibit similar behavior in respect of the laser radiation, so that it is possible to use as further chromophores known recording agents the action of which is synergistically enhanced by the compounds of formula (I) or (II).

When further chromophores or colored stabilizers having optical properties that are as different as possible from those of compounds of formula (I) or (II) are used, they advantageously have an absorption maximum that is hypso-chromically or batho-chromically shifted relative to the metal complex of formula (I) or (II). In that case the absorption maxima are preferably at least 50 nm, especially at least 100 nm, apart.

Examples thereof are UV absorbers that are hypso-chromic to the dye of formula (I) or (II) or colored stabilizers that are bathochromic to the dye of formula (I) or (II) and have absorption maxima lying, for example, in the NIR or IR range.

Other dyes can also be added for the purpose of color-coded identification, color-masking (“diamond dyes”) or enhancing the aesthetic appearance of the recording layer. In all those cases, the further chromophores or colored stabilizers should preferably exhibit behavior towards light and laser radiation that is as inert as possible.

When another dye is added in order to modify the optical properties of the compounds of formula (I) or (II), the amount thereof is dependent upon the optical properties to be achieved. The person skilled in the art will find little difficulty in varying the ratio of additional dye to compound of formula (I) or (II) until he obtains his desired result.

When chromophores or colored stabilizers are used for other purposes, the amount thereof should preferably be small so that their contribution to the total absorption of the recording layer in the range of from 350 to 700 nm is a a maximum of 20%, preferably a maximum of 10%. In such a case, the amount of additional dye or stabilizer is advantageously a maximum of 50% by weight, preferably a maximum of 10% by weight, based on the recording layer.

Most preferably, however, no additional chromophore is added, unless it is a colored stabilizer.

Stabilizers, ¹0₂-, triplet- or luminescence-quenchers are, for example, metal complexes of N- or S-containing enolates, phenolates, bisphenolates, thiolates or bisthiolates or of azo, azomethine or formazan dyes, such as bis(4-dimethylaminodithiobenzil)nickel [CAS No 38465-55.3]. Hindered phenols and derivatives thereof such as o-hydroxyphenyl-triazoles or -triazines or other UV absorbers, such as hindered amines (TEMPO or HALS, as well as nitroxides or NOR-HALS), and also as cations diimmonium, Paraquat™ or Orthoquat salts, such as ®Kayasorb IRG 022, ®Kayasorb IRG 040, optionally also as radical ions, such as N,N,N′,N′-tetrakis(4-dibutylaminophenyl)-p-phenylene amine-ammonium hexafluorophosphate, hexafluoroantimonate or perchlorate. The latter are available from Organica (Wolfen/DE); ®Kayasorb brands are available from Nippon Kayaku Co. Ltd.

The person skilled in the art will know from other optical information media, or will easily identify, which additives in which concentration are best suited to which purpose. Suitable concentrations of additives are, for example, from 0.001 to 1000% by weight, preferably from 1 to 50% by weight.

### (d) Reflecting Layer

Reflecting materials suitable for the reflective layer include especially metals, which provide good reflection of the laser radiation used for recording and playback, for example the metals of Main Groups III, IV and V and of the Sub-groups of the Periodic Table of the Elements. Al, In, Sn, Pb, Sb, Bi, Cu, Ag, Au, Zn, Cd, Hg, Sc, Y, La, Ti, Zr, Hf, V, Nb, Ta, Cr, Mo, W, Fe, Co, Ni, Ru, Rh, Pd, Os, Ir, Pt, Ce, Pr, Nd, Pm, Sm, Eu, Gd, Tb, Dy, Ho, Er, Tm, Yb and Lu and alloys thereof are especially suitable. Special preference is given to a reflective layer of aluminum, silver, copper, gold or an alloy thereof, on account of their high reflectivity and ease of production.

### (e) Cover Layer/Protective Layer

Materials suitable for the cover layer/protective layer include plastics, which are applied in a thin layer to the support or the uppermost layer either directly or with the aid of adhesive layers. It is advantageous to select mechanically and thermally stable plastics having good surface properties, which may be modified further.

The plastics may be thermosetting plastics and thermoplastic plastics. Preference is given to radiation-cured (e.g. using UV radiation) protective layers, which are particularly simple and economical to produce. A wide variety of radiation-curable materials are known. Examples of radiation-curable monomers and oligomers are acrylates and methacrylates of diols, triols and tetrols, polyimides of aromatic tetracarboxylic acids and aromatic diamines having C₁-C₄alkyl groups in at least two ortho-positions of the amino groups, and oligomers with dialkylmaleinimidyl groups, e.g. dimethyl maleinimidyl groups.

The recording media according to the invention may also have additional layers, for example interference layers. It is also possible to construct recording media having a plurality of (for example two) recording layers. The structure and the use of such materials are known to the person skilled in the art. Preferred, if present, are interference layers that are arranged between the recording layer and the reflecting layer and/or between the recording layer and the substrate and consist of a dielectric material, for example as described in EP 0353393 of Ti0₂, Si₃N₄, ZnS or silicone resins.

The recording media according to the invention can be produced by processes known in the art.

### Coating Methods

Suitable coating methods are, for example, immersion, pouring, brush-coating, blade-application and spin-coating, as well as vapor-deposition methods carried out under a high vacuum. When pouring methods are used, solutions in organic solvents are generally used. When solvents are employed, care should be taken that the supports used are insensitive to those solvents. Suitable coating methods and solvents are described, for example, in EP-A-401 791.

The recording layer is preferably applied by spin-coating with a dye solution, solvents that have proved satisfactory are preferably alcohols, e.g. 2-methoxyethanol, n-propanol, isopropanol, isobutanol, n-butanol, amyl alcohol or 3-methyl-1-butanol or preferably fluorinated alcohols, e.g. 2,2,2-trifluoroethanol or 2,2,3,3-tetrafluoro-1-propanol, octafluoropentanol and mixtures thereof. It will be understood that other solvents or solvent mixtures can also be used, for example those solvent mixtures described in EP-A-511 598 and EP-A-833 316. Ethers (dibutyl ether), ketones (2,6-dimethyl-4-heptanone, 5-methyl-2-hexanone) or saturated or unsaturated hydrocarbons (toluene, xylene) can also be used, for example in the form of mixtures (e.g. dibutyl ether/2,6-dimethyl-4-heptanone) or mixed components.

The person skilled in the art of spin-coating will in general routinely try out all the solvents with which is he is familiar, as well as binary and ternary mixtures thereof, in order to discover the solvents or solvent mixtures which result in a high-quality and, at the same time, cost-effective recording layer containing the solid components of his choice. Known methods of process engineering can also be employed in such optimization procedures, so that the number of experiments to be carried out can be kept to a minimum.

The invention therefore relates also to a method of producing an optical recording medium, wherein a solution of a compound of formula (I) or (II) in an organic solvent is applied to a substrate having pits. The application is preferably carried out by spin-coating.

The application of the metallic reflective layer is preferably effected by sputtering, vapor-deposition in vacuum or by chemical vapor deposition (CVD). The sputtering technique is especially preferred for the application of the metallic reflective layer on account of the high degree of adhesion to the support. Such techniques are known and are described in specialist literature (e.g. J. L. Vossen and W. Kern, “Thin Film Processes”, Academic Press, 1978).

### Readout Methods

The structure of the recording medium according to the invention is governed primarily by the readout method; known function principles include the measurement of the change in the transmission or, preferably, in the reflection, but it is also known to measure, for example, the fluorescence instead of the transmission or reflection.

When the recording material is structured for a change in reflection, the following structures, can be used: transparent support/recording layer (optionally multilayered)/reflective layer and, if expedient, protective layer (not necessarily transparent); or support (not necessarily transparent)/reflective layer/recording layer and, if expedient, transparent protective layer. In the first case, the light is incident from the support side, whereas in the latter case the radiation is incident from the recording layer side or, where applicable, from the protective layer side. In both cases the light detector is located on the same side as the light source. The first-mentioned structure of the recording material to be used according to the invention is generally preferred.

When the recording material is structured for a change in light transmission, the following different structure comes into consideration: transparent support/recording layer (optionally multilayered) and, if expedient, transparent protective layer. The light for recording and for readout can be incident either from the support side or from the recording layer side or, where applicable, from the protective layer side, the light detector in this case always being located on the opposite side.

When recording layer is made of dyes of the family described absorbing around 350-450 nm, suitable lasers are those having a wavelength of 350-500 nm, for example commercially available lasers having a wavelength of 405 to 415 nm, especially semi-conductor lasers. When recording layer is made of dyes of the family described absorbing around 550-650 nm, suitable lasers are those having a wavelength of 630-650 nm, for example commercially available lasers having a wavelength of 635 to 640 nm, especially semi-conductor lasers. The recording is done, for example, point for point, by modulating the laser in accordance with the mark lengths and focusing its radiation onto the recording layer. It is known from the specialist literature that other methods are currently being developed which may also be suitable for use.

The process according to the invention allows the storage of information with great reliability and stability, distinguished by very good mechanical and thermal stability and by high light stability and by sharp boundary zones of the pits. Special advantages include the high contrast, the low jitter and the surprisingly high signal/noise ratio, so that excellent readout is achieved.

The readout of information is carried out according to methods known in the art by registering the change in absorption or reflection using laser radiation, for example as described in “CD-Player and R-DAT Recorder” (Claus Biesch-Wiepke, Vogel Buchverlag, Würzburg 1992).

The optical recording medium according to the invention is preferably a recordable optical disc of the WORM type as for example a DVD-R media. It may be also used, for example, as a playable HD-DVD (high density digital versatile disc) or Blu-ray® disc, as storage medium for a computer or as an identification and security card or for the production of diffractive optical elements, for example holograms.

The invention accordingly relates also to a method for the optical recording, storage and playback of information, wherein a recording medium according to the invention is used. The recording and the playback advantageously take place in a wavelength range of from 350 to 500 nm or from 600-650 nm depending on the maximum dye absorbtion.

It has been found, that the new pyridine N-oxide based azo dyes of formula (I) or (II) according to the invention enhance the photosensitivity and the stability to light and heat compared to dyes already known in the art. The new pyridine N-oxide based azo dyes of formula (I) or (II) according to the invention have a decomposition temperature of 250-350° C. Additionally, these compounds show an extremely good solubility in organic solvents, which is ideal for the spin-coating process to manufacture optical layers.

Thus, it is of great advantage to use these new compounds in the recording layer of high-density recordable optical discs.

### EXAMPLES

### Example 1

### Ligands Synthesis

Mono azo dye (1): A mixture of 6.74 g 2-amino-6-methylpyridine N-oxide hydrochloride, 17 ml of water and 17.5 g of concentrated hydrochloric acid (34%) was gradually admixed with 9 ml of a 33% w/v sodium nitrite solution at 0° C.; After 1 hour of reaction at 0° C., the excess of nitrous acid was neutralized with sulfamic acid. The diazotization solution was added dropwise to a solution of 6.31 g of N,N′-dimethylbarbituric acid in water (20 ml) and acetic acid (10 ml) while maintaining pH at 4.2-4.8 with a sodium hydroxide solution (30%). The batch was stirred 3 hours then filtered with suction. The precipitate was washed with water and dried. The presscake yielded 10.7 g of dye ligand of the following formula (I).

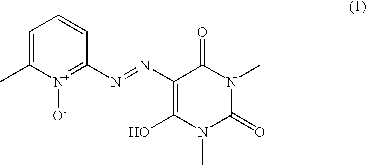

Yield: 79%. λ max (CH₂Cl₂)=411 nm.

### Example 2

### Metal Azo Complex Synthesis

3.36 g of the monoazo dyestuff (1) described in example 1 were suspended in 70 ml of ethanol together with 1.36 g of sodium acetate trihydrate. After heating up to reflux, 1.00 g of copper acetate monohydrate was added. The dyestuff suspension was cooled down to room temperature and the resulting precipitate is stirred for one hour, filtered and the residue washed salt free with deionized water and dried. 3.64 g of the compound (2) with the following formula is obtained.

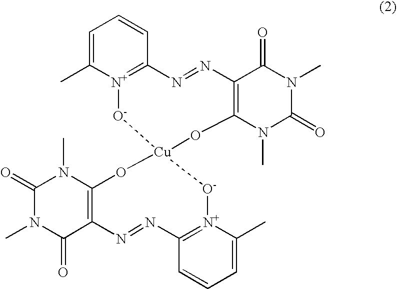

Yield: 99%, λ max (CH₂Cl₂)=417 nm, ε (at 417 nm)=56 l/g·cm. DSC: dec. at 286° C. Solubility in TFP up to 20 g/l.

### Example 3

Dye (3) was prepared as example 2 replacing Cu by Ni.

Yield=99%. λ max (CH₂Cl₂)=433 nm, ε (at 433 nm)=67 l/g·cm. DSC: dec. at 334° C. Solubility in TFP up to 20 g/l.

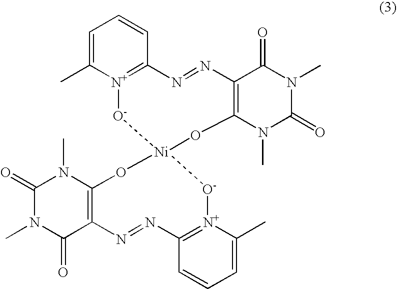

### Example 4

A monoazo dye was prepared according to example 1 using 2-amino-3-ethoxypyridine N-oxide hydrochloride as diazonium precursor (Yield: 74%, λ max (CH₂Cl₂)=404 nm). Then metallisation was performed according to example 2 replacing Cu by Ni. Dye (4) was obtained.

Yield: 92%. λ max (CH₂Cl₂)=435 nm, ε (at 435 nm)=68 l/g·cm. DSC: dec. at 308° C. (30 W/g). Solubility in TFP up to 20 g/l.

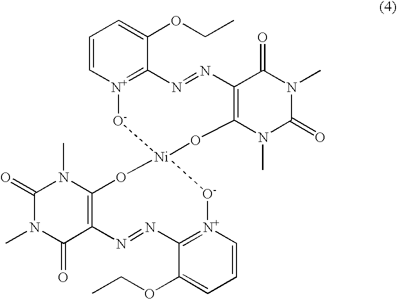

### Example 5

A monoazo dye was prepared according to example 1 using 2-amino-5-bromo-3-ethoxypyridine N-oxide hydrochloride as diazonium precursor (Yield: 80%).

Then metallisation was performed according to example 2 replacing Cu by Ni. Dye (5) was obtained.

Yield: 56%. λ max (CH₂Cl₂)=441 nm, ε (at 441 nm)=60 l/g·cm. DSC: dec. at 271° C. (17 W/g). Solubility in TFP up to 20 g/l.

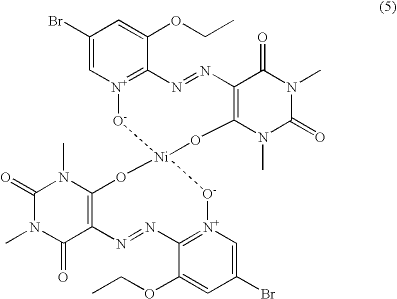

### Example 6

A monoazo dye was prepared according to example 1 using 2-amino-5-bromopyridine N-oxide as diazonium precursor (Yield: 32%).

Then metallisation was performed according to example 2 replacing Cu by Ni. Dye (6) was obtained.

Yield: 82%. λ max (CH₂Cl₂)=438 nm, ε (at 438 nm)=63 l/g·cm. Solubility in TFP up to 20 g/l.

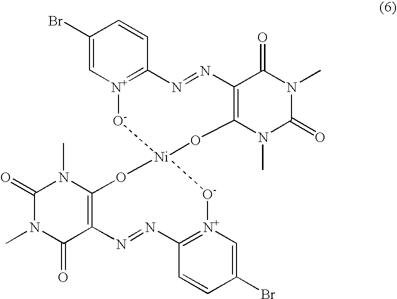

### Example 7

A monoazo dye was prepared according to example 1 using 1-butyl-3-cyano-4-methyl-2-oxo-6-pyridinol as coupling component (Yield: 84%, λ max (CH₂Cl₂)=448 nm). Then metallisation was performed according to example 2. Dye (7) was obtained.

Yield: 78%. λ max (CH₂Cl₂)=463 nm, ε (at 463 nm)=86 l/g·cm. DSC: dec. at 283° C. (65 W/g). Solubility in TFP up to 20 g/l.

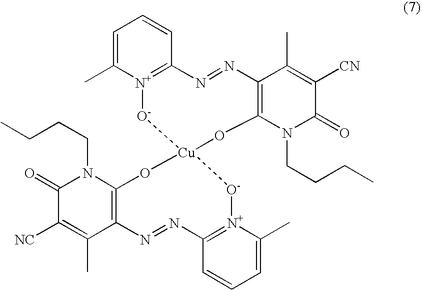

### Example 8

A monoazo dye was prepared according to example 1 using 2-aminopyridine N-oxide hydrochloride as diazonium precursor and 3-methyl-1-phenyl-2-pyrazolin-5-one as coupling component (Yield: 86%).

Then metallisation was performed according to example 2. Dye (8) was obtained.

Yield: 83%. λ max (CH₂Cl₂)=425 nm, ε (at 425 nm)=64 l/g·cm. DSC: dec. at 300° C. (60 W/g).

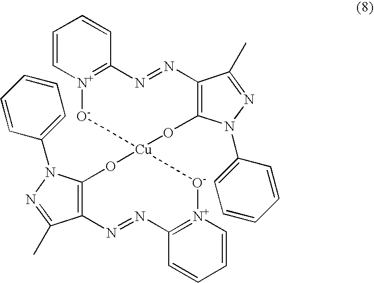

### Example 9

A monoazo dye was prepared according to example 1 using 2-amino-3-ethoxypyridine N-oxide hydrochloride as diazonium precursor and 1,3-diethyl-2-thiobarbituric acid as coupling component (Yield: 85%).

Then metallisation was performed according to example 2 replacing Cu by Ni. Dye (9) was obtained.

Yield: 81%. λ max (CH₂Cl₂)=463 nm, ε (at 463 nm)=76 l/g·cm. Solubility in TFP up to 20 g/l.

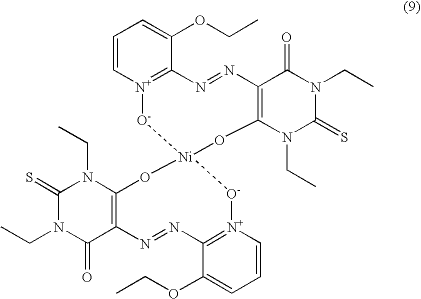

### Example 10

Dye (10) was prepared as in example (9) by replacing Ni by Cu.

Yield: 80%. λ max (CH₂Cl₂)=452 nm, ε (at 452 nm)=65 l/g·cm. DSC: dec. at 240° C. (24 W/g). Solubility in TFP up to 20 g/l.

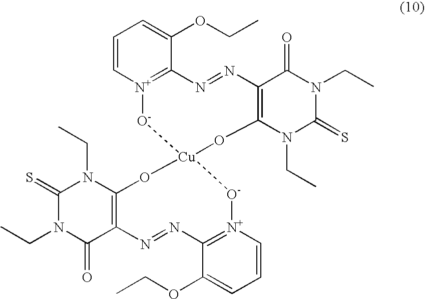

### Example 11

A monoazo dye was prepared according to example 1 using 2-amino-3-ethoxypyridine N-oxide hydrochloride as diazonium precursor and 1-ethyl-6-(dicyanomethylene)-2-pyridinol as coupling component (Yield: 43%).

Then metallisation was performed according to example 2 replacing Cu by Ni. Dye (11) was obtained.

Yield: 85%. λ max (CH₂Cl₂)=560 nm, ε (at 560 nm)=132 l/g·cm. DSC: dec. at 308° C. (7 W/g).

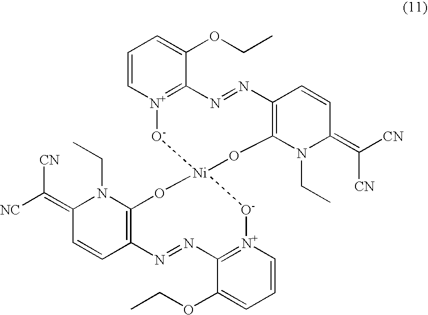

### Example 12

Monoazo dye (12) was prepared according to example 1 using 2-amino-3-hydroxypyridine N-oxide as diazonium precursor and 1-ethyl-6-(dicyanomethylene)-2-pyridinol as coupling component (Yield: 66%).

λ max (CH₂Cl₂)=602 nm, ε (at 602 nm)=105 l/g·cm. DSC: dec. at 260° C. (60 W/g).

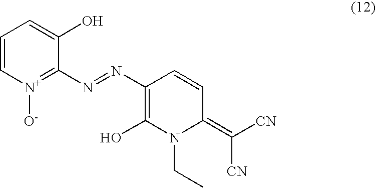

TABLE 1
Summary
Dyes for blue laser recording

| | λ max | ε at λ max | Dec.Pt | Solubility |
|---|---|---|---|---|
| Compound | (nm) | (l/g · cm) | (° C.) | (g/l) |
| (2) | 417 | 56 | 286 | >20 |
| (3) | 433 | 67 | 334 | >20 |
| (4) | 435 | 68 | 308 | >20 |
| (5) | 441 | 60 | 271 | >20 |
| (6) | 438 | 63 | — | >20 |
| (7) | 480 | 107 | 314 | >20 |
| (8) | 425 | 64 | 300 | — |
| (9) | 463 | 76 | — | >20 |
| (10) | 452 | 65 | 240 | >20 |
| (11) | 560 | 132 | 308 | — |
| (12) | 602 | 105 | 260 | — |

*solubility was evaluated in tetrafluoropropanol

### Application Example

The optical and thermal properties of the pyridine N-oxide based azo dye compounds were studied. The dyes show high absorption at the desired wavelengths. In addition, the shape of the absorption spectra, that still remains critical to the disc reflectivity and formation of clean mark edges, are composed of one major band, comprised in a range of from 350 to 700 nm.

More precisely, n values of the refractive index were evaluated between 1.0 and 2.7 (see example 1). Light stabilities were found comparable to commercial dyes which usually are stabilized with quenchers for the use in optical data recording.

Sharp threshold of thermal decomposition within the required temperature range characterizes the new pyridine N-oxide based azo dyes which are assumed to be desirable for the application in optical layers for optical data recording.

As a conclusion, the pyridine N-oxide based azo dye compounds are within the specifications which are primarily required by the industry for the use of dyes in optical data recording, in particular in the next-generation optical data recording media in the blue or red laser range.

1. An optical layer dye compound of formula (I) for an optical layer for optical data recording, 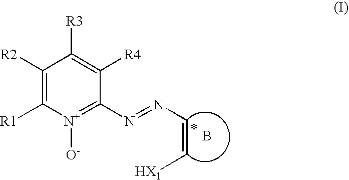
wherein
R₁ to R₄ independently of one another, are hydrogen, cyano (—CN), halogen (F, Cl, Br, I), nitro (NO₂), hydroxy (—OH);
C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkenyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
C₁₋₈ alkoxy (—OR) wherein the alkyl (R) is unsubstituted or substituted by C₁₋₈ alkyl, C₁₋₈ alkenyl, hydroxy (—OH), C₆₋₁₂ aryl or by —NR₅R₆ in which R₅ and R₆ are independently hydrogen, C₁₋₈ alkyl, C₁₋₈ alkenyl or C₆₋₁₂ aryl;
—CX₃ where X is chlorine, fluorine, bromine;
—NR₅R₆ in which R₅ and R₆ are independently hydrogen, C₁₋₈ alkyl, C₁₋₈ alkenyl or C₆₋₁₂ aryl;
C₁₋₈ alkylthio (—SR), wherein the alkyl (R) is unsubstituted or substituted by C₁₋₈ alkyl, hydroxy (—OH), C₆₋₁₂ aryl or by —NR₅R₆ in which R₅ and R₆ are independently hydrogen, C₁₋₈ alkyl or C₆₋₁₂ aryl;
C₁₋₈ sulfoxide (—S(O)R), wherein the alkyl (R) is unsubstituted or substituted by C₁₋₈ alkyl, hydroxy (—OH), C₆₋₁₂ aryl or by —NR₅R₆ in which R₅ and R₆ are independently hydrogen, C₁₋₈ alkyl or C₆₋₁₂ aryl;
C₁₋₈ sulfone (—SO₂R), wherein the alkyl (R) is unsubstituted or substituted by C₁₋₈ alkyl, hydroxy (—OH), C₆₋₁₂ aryl or by —NR₅R₆ in which R₅ and R₆ are independently hydrogen, Cl, alkyl or C₆₋₁₂ aryl;
C₁₋₈ sulfonamide (—SO₂NR₅R₆), in which R₅ and R₆ are independently hydrogen, C₁₋₈ alkyl or C₆₋₁₂ aryl;
C₁₋₈ carbonamide (—CO₂NR₅R₆), in which R₅ and R₆ are independently hydrogen, C₁₋₈ alkyl or C₆₋₁₂ aryl;
C₁₋₈ phosphoramide (—P(O)(NR₅R₆)₂), in which R₅ and R₆ are independently hydrogen, C₁₋₈ alkyl or C₆₋₁₂ aryl;
C₁₋₈ phosphate (—P(O)(OR)₂), in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
X₁ is oxygen, sulphur, selenium,
—NR in which R is hydrogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl; —NSO₂R in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—NP(O)(OR)₂ in which R is C₁₋₄ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆, in which R₅ and R₆ are independently hydrogen, C₁₋₈ alkyl or C₆₋₁₂ aryl;
B is an unsubstituted or a substituted aromatic ring; or
a five or a six-membered heterocyclic ring of one of the following structures: 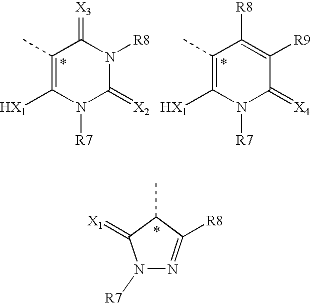
wherein
R₇ to R₉ independently of one another, are hydrogen, halogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl; —SO₂R wherein R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
X₂ to X₃ are, independently, oxygen, sulphur or selenium, —NR in which R is hydrogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—NSO₂R in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—NP(O)(OR)₂ in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
X₄ is oxygen, sulphur, selenium;
═NR in which R is hydrogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl; or
═C(CN)₂;
M is a metal atom selected from the group consisting of Al, In, Sn, Ti, V, Cr, Mn, Fe, Co, Ni, Cu, Zn, Zr, Ru, Rh, Pd, Cd, Hf, Re, Os, Ir, Pt, Hg.

2. The optical layer dye compound according to claim 11, wherein the dye compound is of formula (II), and wherein the dye compound of formula (II) is of the formula (III) 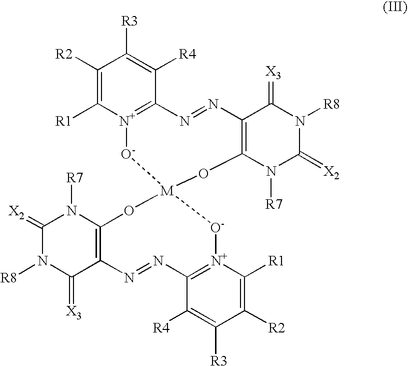
wherein
M is selected from the group consisting of Ni, Cu, Co, Zn, Al, Fe, Ru, Pd, Pt, Cr, and Mn;
R₁ is H, Cl, CH₃, C₂H₅, C₃H₇ or unsubstituted phenyl,
R₂ is H, Br, Cl, F or NR₂ wherein the alkyl (R) is unsubstituted or substituted by C₁₋₈ alkyl, hydroxy (—OH), C₆₋₁₂ aryl or by —NR₇R₈ in which R₇ and R₈ are independently hydrogen, C₁₋₈ alkyl, C₆₋₁₂ aryl; NO₂, CH₃ or C₂H₅,
R₃ is H, Cl, CH₃ or C₂H₅,
R₄ is hydrogen, CH₃, C₂H₅ or OR wherein the alkyl (R) is unsubstituted or substituted by C₁₋₈ alkyl, hydroxy (—OH), C₆₋₁₂ aryl or by —NR₇R₈ in which R₇ and R₈ are independently hydrogen, C₁₋₈ alkyl or C₆₋₁₂ aryl;
R₇ to R₈ independently of one another, are hydrogen, C₁₋₈ alkyl alkenyl, alkynyl, C₃₋₁₀ cycloalkyl or cycloalkenyl wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
X₂ to X₃ is O, S or NR wherein R is hydrogen, cyano, or C₁₋₈ alkyl.

3. The optical layer dye compound according to claim 2, wherein
M is selected from the group consisting of Ni, Cu, Co, Zn, Al, Fe, Cr and Mn;
R₁ is H or CH₃,
R₂ is H, Br, Cl, NO₂ or CH₃,
R₃ is hydrogen,
R₄ is H, Cl, CH₃, OC₂H₅ or OH,
R₇ to R₈ independently of one another, are CH₃, C₂H₅, C₃H₇ (n- or i-propyl) or C₄H₉ (n-, sek- or tert.-butyl),
X₂ is O or S.
X₃ is O.

4. The optical layer dye compound according to claim 3,
wherein
M is nickel, copper or zinc,
R₁ is H,
R₂ is Br,
R₃ is hydrogen,
R₄ is OC₂H₅,
R₇ and R₈ are C₂H₅, and
X₂ is S,
X₃ is O.

5. An optical layer comprising at least one optical layer dye compound according to formula (I) as defined in claim 1 or a mixture of at least two optical layer dye compounds according to formula (I) as defined in claim 1.

6. A method for producing an optical layer comprising the steps of
(a) providing a substrate
(b) dissolving an optical layer dye compound or a mixture of optical layer dye compounds of formula (I) as claimed in claim 1 in an organic solvent to form a solution,
(c) coating the solution (b) on the substrate (a);
(d) evaporating the solvent to form a dye layer.

7. A method according to claim 6, wherein the substrate is polycarbonate (PC) or polymethylmethacrylate (PMMA).

8. A method according to claim 6, wherein the organic solvent is selected from the group consisting of C₁₋₈ alcohol, halogen substituted C₁₋₈ alcohols, C₁₋₈ ketone, C₁₋₈ ether, halogen substituted C₁₋₈alkane, and amides.

9. A method according to claim 8, wherein the C₁₋₈ alcohols or halogen substituted C₁₋₈ alcohols are selected from the group consisting of methanol, ethanol, isopropanol, diacetone alcohol (DM), 2,2,3,3-tetrafluoropropanol, trichloroethanol, 2-chloroethanol, octafluoropentanol (wand hexafluorobutanol; the C₁₋₈ ketones are selected from the group consisting of acetone, methylisobutylketone, methylethylketone, and 3-hydroxy-3-methyl-2-butanone; the halogen substituted C₁₋₄ alkanes are selected from the group consisting of chloroform, dichloromethane and 1-chlorobutane; and the amides are dimethylformamide or dimethylacetamide.

10. An optical recording medium comprising an optical layer according to claim 5.

11. An optical layer dye compound of formula (II) for an optical layer for optical data recording, 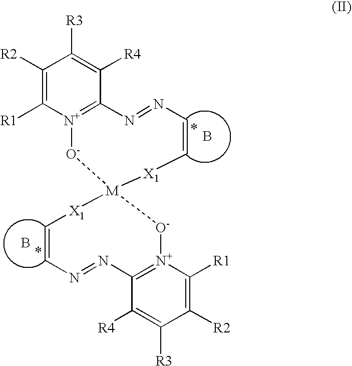
wherein
R₁ to R₄ independently of one another, are hydrogen, cyano (—CN), halogen (F, Cl, Br, I), nitro (NO₂), hydroxy (—OH);
C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkenyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
C₁₋₈ alkoxy (—OR), wherein the alkyl (R) is unsubstituted or substituted by C₁₋₈ alkyl, C₁₋₈ alkenyl, hydroxy (—OH), C₆₋₁₂ aryl or by —NR₅R₆ in which R₅ and R₆ are independently hydrogen, C₁₋₈ alkyl, C₁₋₈ alkenyl or
C₆₋₁₂ aryl;
—CX₃ where X is chlorine, fluorine, bromine;
—NR₅R₆ in which R₅ and R₆ are independently hydrogen, C₁₋₈ alkyl, C₁₋₈ alkenyl or C₆₋₁₂ aryl;
C₁₋₈ alkylthio (—SR), wherein the alkyl (R) is unsubstituted or substituted by C₁₋₈ alkyl, hydroxy (—OH), C₆₋₁₂ aryl or by —NR₅R₆ in which R₅ and R₆ are independently hydrogen, C₁₋₈ alkyl or C₆₋₁₂ aryl;
C₁₋₈ sulfoxide (—S(O)R), wherein the alkyl (R) is unsubstituted or substituted by C₁₋₈ alkyl, hydroxy (—OH), C₆₋₁₂ aryl or by —NR₅R₆ in which R₅ and R₆ are independently hydrogen, C₁₋₈ alkyl or C₆₋₁₂ aryl;
C₁₋₈ sulfone (—SO₂R), wherein the alkyl (R) is unsubstituted or substituted by C₁₋₈ alkyl, hydroxy (—OH), C₆₋₁₂ aryl or by —NR₅R₆ in which R₅ and R₆ are independently hydrogen, Cl, alkyl or C₆₋₁₂ aryl;
C₁₋₈ sulfonamide (—SO₂NR₅R₆), in which R₅ and R₆ are independently hydrogen, C₁₋₈ alkyl or C₆₋₁₂ aryl;
C₁₋₈ carbonamide (—CO₂NR₅R₆), in which R₅ and R₆ are independently hydrogen, C₁₋₈ alkyl or C₆₋₂ aryl;
C₁₋₈ phosphoramide (—P(O)(NR₅R₆)₂), in which R₅ and R₆ are independently hydrogen, C₁₋₈ alkyl or C₆₋₁₂ aryl;
C₁₋₈ phosphate (—P(O)(OR)₂), in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁ alkoxy, halogen, hydroxy
(—OH), C₆₋₁₂ aryl or by —NR₅R₆ in which R₅ and R₆ are independently hydrogen, C₁₋₈ alkyl or C₆₋₁₂ aryl;
X₁ is oxygen, sulphur, selenium,
—NR in which R represent hydrogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl; —NSO₂R in which R represent C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—NP(O)(OR)₂ in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
B is an unsubstituted or a substituted aromatic ring; or
a five or a six-membered heterocyclic ring of one of the following structures: 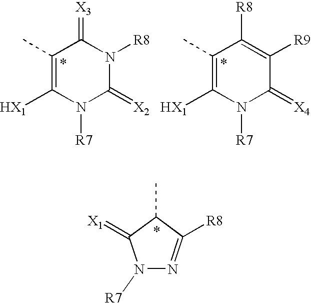
wherein
R₇ to R₉ independently of one another, are hydrogen, halogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—SO₂R wherein R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
X₂ to X₃ are, independently oxygen, sulphur, selenium,
—NR in which R represent hydrogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—NSO₂R in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
—NP(O)(OR)₂ in which R is C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈ alkyl or C₆₋₁₂ aryl;
X₄ is oxygen, sulphur, selenium;
═NR in which R is hydrogen, cyano, C₁₋₈ alkyl, alkenyl or alkynyl, C₃₋₁₀ cycloalkyl or cycloalkenyl, wherein the alkyl groups are unsubstituted or substituted by C₁₋₈ alkyl, C₁₋₈ alkoxy, halogen, hydroxy (—OH), C₆₋₁₂ aryl or by —NR₅R₆ in which R₅ and R₆ are independently hydrogen, C₁₋₈alkyl or C₆₋₁₂ aryl; or
═C(CN)₂;
M is a metal atom selected from the group consisting of Al, In, Sn, Ti, V, Cr, Mn, Fe, Co, Ni, Cu, Zn, Zr, Ru, Rh, Pd, Cd, Hf, Re, Os, Ir, Pt and Hg.

12. An optical layer comprising at least one optical layer dye compound according to formula (II) as defined in claim 11, or a mixture of at least two optical layer dye compounds according to formula (II) as defined in claim 11.

13. A method for producing an optical layer comprising the steps of
(a) providing a substrate
(b) dissolving an optical layer dye compound or a mixture of optical layer dye compounds of formula (I) as claimed in claim 11 in an organic solvent to form a solution,
(c) coating the solution (b) on the substrate (a);
(d) evaporating the solvent to form a dye layer.

14. A method according to claim 13, wherein the substrate is polycarbonate (PC) or polymethylmethacrylate (PMMA).

15. A method according to claim 13, wherein the organic solvent is selected from the group consisting of Cl, alcohol, halogen substituted C₁₋₈ alcohols, C₁₋₈ ketone, C₁₋₈ ether, halogen substituted C₁₋₄ alkane, and amides.

16. A method according to claim 15, wherein the C₁₋₈ alcohols or halogen substituted C₁₋₈ alcohols are selected from the group consisting of methanol, ethanol, isopropanol, diacetone alcohol (DAA), 2,2,3,3-tetrafluoropropanol, trichloroethanol, 2-chloroethanol, octafluoropentanol and hexafluorobutanol;
the C₁₋₈ ketones are selected from the group consisting of acetone, methylisobutylketone, methylethylketone, and 3-hydroxy-3-methyl-2-butanone;
the halogen substituted C₁₋₄ alkanes are selected from the group consisting of chloroform, dichloromethane and 1-chlorobutane; and
the amides are dimethylformamide or dimethylacetamide.

17. An optical recording medium comprising an optical layer according to claim 12.
